(19) United States
(12) Patent Application Publication
(10) Pub. No.: US 2022/0402961 A1
(43) Pub. Date: Dec. 22, 2022

(54) NICOTINAMIDE MONONUCLEOTIDE-ISONICOTINE COCRYSTAL AND COMPOSITION THEREOF

(71) Applicants: BONTAC BIO-ENGINEERING(SHENZHEN) CO.,LTD, Shenzhen (CN); ZHONGSHAN BONTAC BIO-TECHNOLOGY CO. , LTD, Zhongshan (CN)

(72) Inventors: Zhang ZHANG, Shenzhen (CN); Ming CHEN, Shenzhen (CN)

(21) Appl. No.: 17/833,110

(22) Filed: Jun. 6, 2022

(30) Foreign Application Priority Data
Jun. 18, 2021 (CN) 2021106777841

(51) Int. Cl. C07H 19/048

(52) U.S. Cl. CPC C07H 19/048; C07B 2200/13

(57) ABSTRACT

The present disclosure aims to solve the technical problems of larger content/weight difference and inconsistent quality of nicotinamide mononucleotide (NMN) medicines or health care products due to poor fluidity of existing nicotinamide mononucleotide crystals, and provides a nicotinamide mononucleotide-isonicotine cocrystal. Cu-Kαradiation is used for the cocrystal, and X-ray powder diffraction represented by an angle 2θ has diffraction peaks at 9.6±0.2°, 13.3±0.3°, 22.8±0.2° and 36.5±0.2°. The cocrystal has a higher bulk density than existing crystals, thereby significantly improving the fluidity of the nicotinamide mononucleotide. Therefore, the technical problems of larger content/weight difference and inconsistent quality of the NMN medicines or health care products in the production of enterprises may be well solved.

### TECHNICAL FIELD

The present disclosure relates to the technical field of compound crystals, in particular to a nicotinamide mononucleotide cocrystal and a composition containing the same.

### BACKGROUND

Nicotinamide mononucleotide (NMN for short) is a biochemical substance inherent in biological cells. It may be adenylated by nicotinamide nucleotide adenosyltransferase in the cells to form an important substance-nicotinamide adenine dinucleotide (NAD for short, also known as coenzyme I, existing in all the cells, taking part in thousands of biocatalytic reactions, and playing an important role in the generation of biological cell energy) that the biological cells depend on for survival. NMN is a direct precursor of NAD. As an important intermediate of an NAD salvage synthesis pathway in biological cells, its level in the biological cells directly affects the concentration of NAD.

Studies have found that supplementing NMN in vitro is a most ideal way to increase the concentration of NAD in cells. In addition, it has also been found that supplementing NMN in vitro may achieve many health care effects of delaying aging, treating Parkinson's and other geriatric diseases, regulating insulin secretion, affecting mRNA expression and the like. Besides, more and more new medical uses of NMN are being reported. In addition, with the news of Li Ka-shing's investment in “elixir” NMN, the NMN has become a favorite for a while and has been favored by many capitals, and the general public is also rushing to pursue NMN medicines or health care products. As a result, the demand for the NMN medicines or health care products is increasing day by day.

Because the stability of NMN is not good enough, medicines or health care products produced from NMN amorphous powder easily lose their pharmaceutical activity during the storage and transportation. Therefore, NMN crystals were developed. For example, two crystal forms of β-nicotinamide mononucleotide were published in Chinese patent application CN108697722A, which are anhydrous crystals (form 1) and dimethyl sulfoxide solvate crystals (form 2) respectively. Nowadays, related enterprises generally adopt NMN in a crystal form to produce NMN medicines or health care products, and the stability of the products has been significantly improved. However, there are still certain problems, such as larger content/weight difference and inconsistent quality of products with poorer fluidity of NMN.

### SUMMARY

In view of the deficiencies mentioned in the above background, an objective of the present disclosure is to solve the technical problems of larger content/weight difference and inconsistent quality of NMN medicines or health care products due to poor fluidity of existing nicotinamide mononucleotide crystals.

To achieve the above objective, the present disclosure provides a nicotinamide mononucleotide-isonicotine cocrystal, wherein Cu-Kαradiation is used for the cocrystal, and X-ray powder diffraction represented by an angle 2θ has diffraction peaks at 9.6±0.2°, 13.3±0.3°, 22.8±0.2° and 36.5±0.2°.

The so-called cocrystal, according to the definition in the “Guidelines (2011) for the Regulatory Classification of Pharmaceutical Cocrystals” published by the Food and Drug Administration (FDA), refers to a crystalline substance containing two or more different molecules in a same crystal lattice. Cocrystal components interact through non-ionization and are in a neutral state. There are two types of cocrystal components, one is an active pharmaceutical ingredient (API for short), and the other is a cocrystal former (CCF for short). Under the action of hydrogen bonds, π-π stacking, Van der Waals forces or other non-covalent bonds, the two types of cocrystal components are combined in a fixed stoichiometric ratio to generate a new solid form.

The inventor has finally developed a new crystal form of nicotinamide mononucleotide, presented in a cocrystal form, provided by the present disclosure through a large amount of long-term experimental exploration and creative work. Experiments find that the nicotinamide mononucleotide-isonicotine cocrystal provided by the present disclosure not only does not affect the pharmaceutical activity of nicotinamide mononucleotide, but has a higher bulk density than existing crystals, thereby significantly improving the fluidity of the nicotinamide mononucleotide.

Further, for the nicotinamide mononucleotide-isonicotine cocrystal provided by the present disclosure, Cu-Kαradiation is used, and X-ray powder diffraction represented by an angle 2θ has diffraction peaks at about 9.6±0.2°, about 9.8±0.2°, about 10.6±0.2°, about 13.3±0.3°, about 16.3±0.2°, about 21.3±0.2°, about 22.8±0.2°, about 32.1±0.2° and about 36.5±0.2°.

Furthermore, the nicotinamide mononucleotide-isonicotine cocrystal provided by the present disclosure has an X-ray powder diffraction spectrum as shown in FIG. 1.

A differential scanning calorimetry analysis diagram of the nicotinamide mononucleotide-isonicotine cocrystal provided by the present disclosure has endothermic peaks at 55.8±3° C. and 151.9±3° C.

Furthermore, the nicotinamide mononucleotide-isonicotine cocrystal provided by the present disclosure has a differential scanning calorimetry analysis diagram as shown in FIG. 2.

In addition, the present disclosure further provides a nicotinamide mononucleotide-isonicotine composition, wherein the composition contains the nicotinamide mononucleotide-isonicotine cocrystal provided by the present disclosure.

Finally, the present disclosure further provides a medicine or health care product, wherein an active pharmaceutical ingredient of the medicine or health care product contains the nicotinamide mononucleotide-isonicotine cocrystal provided by the present disclosure. The medicine or health care product may be a tablet or capsule, and the active pharmaceutical ingredient in the medicine or health care product is the above nicotinamide mononucleotide-isonicotine cocrystal provided by the present disclosure or a combination of the above nicotinamide mononucleotide-isonicotine cocrystal provided by the present disclosure and other active pharmaceutical ingredients.

### The Beneficial Effects:

1. Compared with the prior art, the present disclosure provides a new crystal form of nicotinamide mononucleotide presented in a cocrystal form, which fills the gap of the nicotinamide mononucleotide cocrystal. 2. Most importantly, experiments find that the nicotinamide mononucleotide-isonicotine cocrystal provided by the present disclosure not only does not affect the pharmaceutical activity of nicotinamide mononucleotide, but has a higher bulk density than existing crystals, thereby significantly improving the fluidity of the nicotinamide mononucleotide. Therefore, the technical problems of larger content/weight difference and inconsistent quality of NMN medicines or health care products in the production of enterprises may be well solved.

### BRIEF DESCRIPTION OF THE DRAWINGS

FIG. 1 is an X-ray powder diffraction spectrum of a nicotinamide mononucleotide-isonicotine cocrystal provided by the present disclosure; and

FIG. 2 is a differential scanning calorimetry analysis diagram of the nicotinamide mononucleotide-isonicotine cocrystal provided by the present disclosure.

### DETAILED DESCRIPTION OF THE EMBODIMENTS

The present disclosure is further described in detail below with reference to the accompanying drawings and specific examples. The following examples are to explain the present disclosure. The present disclosure is not limited to the following examples.

Raw materials and reagents used in the following examples were all purchased from the market, unless otherwise specified.

With reference to a method disclosed by an example 1 in Chinese patent application CN108697722A, a nicotinamide mononucleotide anhydrous crystal (form 1) was prepared.

With reference to a method disclosed by an example 4 in Chinese patent application CN108697722A, a nicotinamide mononucleotide dimethyl sulfoxide solvate crystal (form 2) was prepared.

### Example 1

Preparation of the nicotinamide mononucleotide-isonicotine cocrystal provided by the present disclosure

67 g of β-nicotinamide mononucleotide and 24 g of isonicotine were dissolved in 2 L of water, then 2 L of acetone was slowly dropwise added, and stirring was performed while dropwise adding. During the dropwise adding, the temperature of a solution was kept at 30° C. After dropwise adding, the temperature of the solution was lowered to 8° C., and standing was performed to wait for crystals to be precipitated. After crystal precipitation, the solution was filtered to obtain the nicotinamide mononucleotide-isonicotine cocrystal.

The nicotinamide mononucleotide-isonicotine cocrystal prepared above was subjected to X-ray powder diffraction:

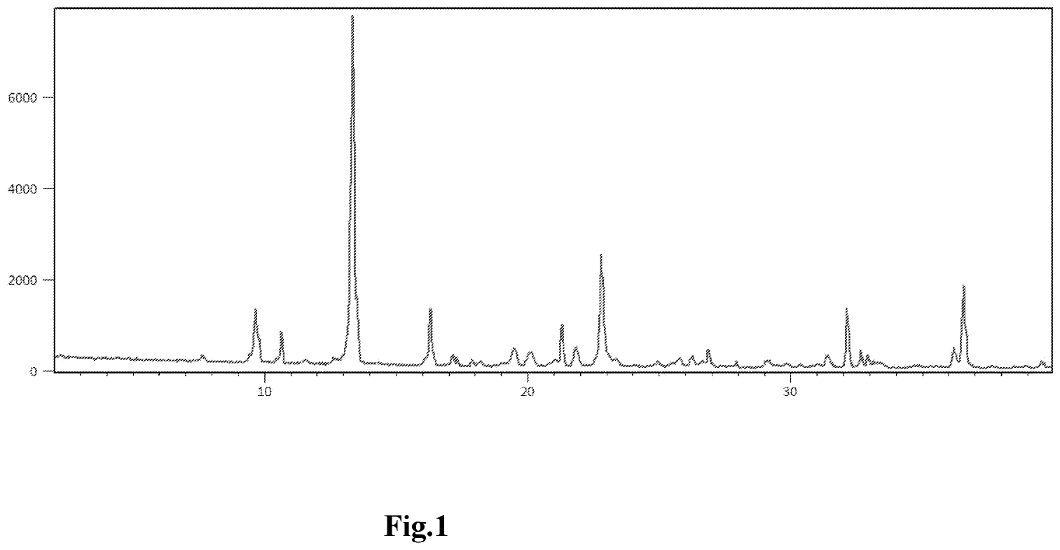

A PANalytical X′Pert Empyrean X-ray powder diffractometer (PW3040/60, Dutch PANalytical Analytical Instruments Ltd.) was used, wherein Cu-Kαradiation is adopted, a wavelength is 1.54 Å, a divergence slit is 1°, an X-ray tube voltage is 45 kV, an X-ray tube current is 40 mA, a scanning range is 2-40° (2θ), a step length is 0.0130°, and step time is 78.7950 s. A powder sample was flattened on a microsample plate and then tested. An X-ray powder diffraction spectrum of the nicotinamide mononucleotide-isonicotine cocrystal provided by the present disclosure is as shown in FIG. 1. Peaks and intensities corresponding to a diffraction angle 2θ are as shown in a table 1.

TABLE 1

| | Relative |
|---|---|
| 2θ (°) | intensity (%) |
| 9.6 | 15.3 |
| 9.8 | 6.8 |
| 10.6 | 8.9 |
| 13.3 | 100.0 |
| 16.3 | 16.5 |
| 17.1 | 3.1 |
| 19.4 | 4.7 |
| 20.1 | 4.3 |
| 21.3 | 11.9 |
| 21.8 | 5.5 |
| 22.8 | 28.2 |
| 25.7 | 2.5 |
| 26.2 | 3.2 |
| 26.8 | 4.9 |
| 31.4 | 3.7 |
| 32.1 | 15.3 |
| 32.6 | 4.6 |
| 32.9 | 3.5 |
| 36.2 | 6.0 |
| 36.5 | 23.4 |

The nicotinamide mononucleotide-isonicotine cocrystal prepared above was subjected to differential scanning calorimetric (DSC) curve measurement:

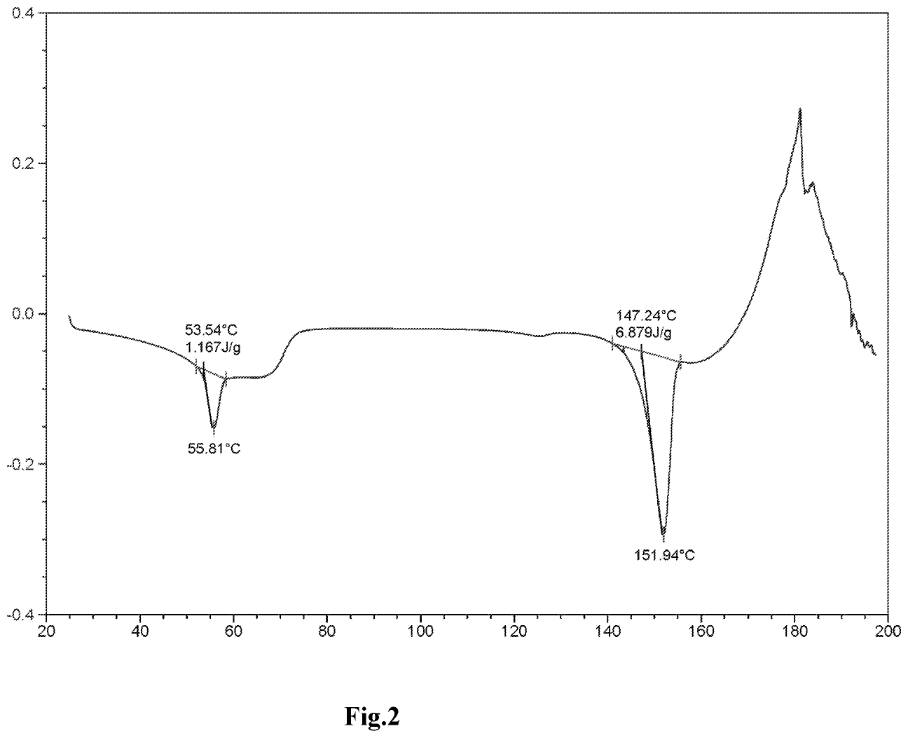

DSC measurement was performed with a seal plate device in a TA Instruments Q2000. A sample (about 1-3 mg) was weighed in an aluminum plate, capped with a Tzero, accurately recorded to 1/100 mg, and transferred to an instrument for measurement. The instrument was purged with nitrogen at 50 mL/min. Data was collected between room temperature and 220° C. at a heating rate of 10° C./min. Endothermic peaks were plotted downwards, and the data was analyzed with TA Universal Analysis. A differential scanning calorimetry analysis diagram of the nicotinamide mononucleotide-isonicotine cocrystal provided by the present disclosure is as shown in FIG. 2, wherein an abscissa represents the temperature (Temperature, ° C.), and an ordinate represents the heat flow (Heat Flow, W/g) released by a substance per unit mass.

### Example 2

Bulk Density Measurement

A proper amount of samples of a crystal in a form 1, a crystal in a form 2 and the nicotinamide mononucleotide-isonicotine cocrystal prepared in the example 1 were taken respectively, screened with a sieve (1.00 mm, No. 18), accurately weighed, and slowly poured into a glass graduated measuring cylinder. The tops were scraped flat. The apparent volumes were recorded. The bulk densities were calculated. Experimental results are as shown in a table 2.

TABLE 2

| Crystal | Bulk density g/ml |
|---|---|
| Form 1 | 0.15 |
| Form 2 | 0.22 |
| Cocrystal in example 1 | 0.68 |

### Example 3

Content Difference Measurement

A proper amount of a crystal in a form 1, a crystal in a form 2 and the nicotinamide mononucleotide-isonicotine cocrystal prepared in the example 1 were taken respectively and screened with a 200-mesh sieve. A capsule shell was fixed to a capsule board. A body board was filled with powder. The powder was poured on the body board and scraped back and forth with a powder scraping plate. After the capsule shell was filled up with the powder, the excess powder on the body board was scraped off to obtain a capsule. Then the capsule obtained by filling was measured with reference to an inspection method for [symbol] content difference [symbol] of capsules in the 0103 capsule general principle of the “Pharmacopoeia of the People's Republic of China” (2020 edition). A content difference value X (%) of the content of each capsule corresponding to each group of the crystal in the form 1, the crystal in the form 2 and the cocrystal in the example 1 and average content after comparison was calculated respectively. Then an absolute value of each content difference value was taken. An average value X̅ of each group was calculated.

$$\overline{X} = \frac{X 1 + X 2 + … Xn}{n} .$$

Results are as shown in a table 3.

TABLE 3

| | Average content |
|---|---|
| Crystal | difference [symbol] (%) |
| Form 1 | 28.8 |
| Form 2 | 26.0 |
| Cocrystal in example 1 | 5.5 |

[symbol]

indicates data missing or illegible when filed

1. A nicotinamide mononucleotide-isonicotine cocrystal, wherein Cu-Kαradiation is used, and X-ray powder diffraction represented by an angle 2θ has diffraction peaks at 9.6±0.2°, 13.3±0.3°, 22.8±0.2° and 36.5±0.2°.

2. The nicotinamide mononucleotide-isonicotine cocrystal according to claim 1, wherein the Cu-Kαradiation is used, and the X-ray powder diffraction represented by the angle 2θ has diffraction peaks at 9.6±0.2°, 9.8±0.2°, 10.6±0.2°, 13.3±0.3°, 16.3±0.2°, 21.3±0.2°, 22.8±0.2°, 32.1±0.2° and 36.5±0.2°.

3. The nicotinamide mononucleotide-isonicotine cocrystal according to claim 1, wherein the cocrystal has an X-ray powder diffraction spectrum substantially as shown in FIG. 1.

4. The nicotinamide mononucleotide-isonicotine cocrystal according to claim 1, wherein a differential scanning calorimetry analysis diagram of the cocrystal has endothermic peaks at 55.8±3° C. and 151.9±3° C.

5. The nicotinamide mononucleotide-isonicotine cocrystal according to claim 1, wherein the cocrystal has a differential scanning calorimetry analysis diagram substantially as shown in FIG. 2.

6. A nicotinamide mononucleotide composition, wherein the composition contains the nicotinamide mononucleotide-isonicotine cocrystal according to claim 1.

7. A medicine or health care product, wherein an active pharmaceutical ingredient of the medicine or health care product contains the nicotinamide mononucleotide-isonicotine cocrystal according to claim 1.
